(19) United States
(12) Patent Application Publication
(10) Pub. No.: US 2019/0191710 A1
(43) Pub. Date: Jun. 27, 2019

(54) MIXTURES OF SABADILLA ALKALOIDS WITH LYSINIBACILLUS SPHAERICUS OR MIXTURES OF SABADILLA ALKALOIDS WITH BACILLUS THURINGIENSIS AND USES THEREOF

(71) Applicant: McLaughlin Gormley King Company, Golden Valley, MN (US)

(72) Inventor: Robert A. Suranyi, Minneapolis, MN (US)

(21) Appl. No.: 16/226,994

(22) Filed: Dec. 20, 2018

Related U.S. Application Data
(60) Provisional application No. 62/610,766, filed on Dec. 27, 2017.

(51) Int. Cl. A01N 63/04; A01N 43/42; C12N 1/20

(52) U.S. Cl. CPC A01N 63/04; C12N 1/20; A01N 43/42

(57) ABSTRACT

The present invention is directed to pesticidal mixtures comprising sabadilla alkaloids and a bacterium selected from the group consisting of Lysinibacillus sphaericus and a mixture of Lysinibacillus sphaericus and Bacillus thuringiensis and methods of controlling pests by application of pesticidal mixtures comprising sabadilla alkaloids and a bacterium selected from the group consisting of Lysinibacillus sphaericus and a mixture of Lysinibacillus sphaericus and Bacillus thuringiensis.

### FIELD OF THE INVENTION

The present invention is directed to pesticidal mixtures comprising sabadilla alkaloids and a bacterium selected from the group consisting of Lysinibacillus sphaericus and a mixture of Lysinibacillus sphaericus and Bacillus thuringiensis and methods of controlling pests by application of pesticidal mixtures comprising sabadilla alkaloids and a bacterium selected from the group consisting of L. sphaericus and a mixture of L. sphaericus and B. thuringiensis.

### BACKGROUND OF THE INVENTION

Arthropod pests, including insects, are one of the major threats to human welfare and transmit a broad array of medical and veterinary diseases. Synthetic insecticides played a significant role and in many ways ushered in modern agriculture and pest control. However, the widespread use of synthetic insecticides also created numerous environmental challenges. The acute effects of synthetic pesticides on professional applicators and other end users are well-known but the chronic long term human health effects can be equally serious. Further, the use of synthetic insecticides has led to the development of resistant insect populations. Insecticide resistance is a complex phenomenon underlined by a diverse array of physiological mechanisms. Major mechanisms that are responsible for the development of insecticide resistance are metabolic detoxification, target site mutation, reduced cuticular penetration and behavioral avoidance.

Integrated Pest Management (“IPM”) is a holistic approach to pest management. A fundamental aspect of insecticide utilization under the broader framework of IPM is the management of insecticide resistance (IRM) by the utilization of insecticide combinations that reduce the rate of resistance development. A combination of insecticides with different modes of action is fundamentally a concept based upon the idea of redundant killing of target insect populations. Insect within the population adapted to one of the active ingredient in the combination product will still be killed by the other active ingredient. This combination effect will result in an overall greater reduction in population size and be more likely to cause eradication of the entire population. Mixtures can also reduce the amount of pesticides applied in the environment and the environmental impact associated with pesticide applications.

Most botanical insecticides are readily biodegradable and significantly less harmful to the environment and users than synthetic insecticides. The very short environmental persistence, usually less than 24 hours, of plant derived insecticides is favorable to the survival of non-target, beneficial parasites and predators which are important components of IPM. Unlike conventional insecticides which are typically based on a single active ingredient, plant derived insecticides usually comprise an array of chemical compounds that affect both behavioral and physiological functions of the target arthropods. The probability of pest resistance developing to plant derived insecticides is less than that for synthetic pesticides because these mixtures may have a variety of modes of action.

Nematodes, better known as roundworms and more specifically hookworms, pinworms, heart worms etc., are found all over the earth in almost every environment. In fact, nematodes account for about 80% of all individual animals on earth. Over half of nematode species are parasitic and present a significant problem to both plant and animal health.

One effective naturally derived pesticide is found in the tissues of many of the plants of the genus Schoenocaulon, commonly referred to as sabadilla. The species with the longest history of use, and the most readily available, is Schoenocaulon officinale. The plant is indigenous to Central and South America and its seeds have been used for centuries for their insecticidal properties. The seeds contain several alkaloids including veratridine and cevadine, both of which are known to be active against arthropods.

Lysinibacillus sphaericus (previously known as Bacillus sphaericus) is commonly found in the soil. L. sphaericus has been demonstrated to have larvicidal effect on two genera of mosquitoes (Culex and Anopheles). Further, L. sphaericus has been shown to have a nematocidal effect. Specific toxins produced by L. sphaericus include binary toxin (BinA/BinB), mosquitocidal toxin (“Mtx”)1, Mtx2 and Mtx3. While L. sphaericus is effective against some mosquitoes it is not effective against Aedes aegypti, known to carry the viral diseases, yellow fever, dengue and Zika.

Bacillus thuringiensis is a natural soil bacterium. Many Bacillus thuringiensis strains produce crystal proteins during sporulation called δ-endotoxins which can be used as biological insecticides. Bacillus thuringiensis produces crystals which bind to midgut epithelium of insect larvae and initiate a cascade of effects that directly kill the exposed larvae. Binding creates pores and leading to loss of the transmembrane potential, cell lysis, leakage of the midgut contents, paralysis, and death of the insect by septicemia. One advantage of using Bacillus thuringiensis is that they are target specific. They do not harm humans or other non-target species. Yet another advantage of Bacillus thuringiensis is that they can be used on organic crops. Further, with no mandated pre-harvest interval, it can also be used on crops right before harvest.

Bacillus thuringiensis subsp. aizawai is commercially available as XenTari® (available from Valent BioSciences Corporation, XenTari is a registered trademark of Valent BioSciences Corporation). Bacillus thuringiensis subsp. kurstaki is commercially available as Dipel® (available from Valent BioSciences Corporation, Dipel is a registered trademark of Valent BioSciences Corporation). Bacillus thuringiensis subsp. thuringiensis is commercially available as Novodor (available from Valent BioSciences Corporation).

Thus, there is a need in the art for pesticide combinations that contain pesticides that decrease the development of pesticide resistance.

### SUMMARY OF THE INVENTION

In one aspect, the present invention is directed to pesticidal mixtures of sabadilla alkaloids and a bacterium selected from the group consisting of Lysinibacillus sphaericus and a mixture of L. sphaericus and Bacillus thuringiensis.

In another aspect, the present invention is directed to pesticidal mixtures of sabadilla alkaloids and a fermentate of a bacterium selected from the group consisting of L. sphaericus and a mixture of L. sphaericus and B. thuringiensis.

In another aspect, the present invention is directed to pesticidal mixtures of sabadilla alkaloids and toxins produced by a bacterium selected from the group consisting of L. sphaericus and a mixture of L. sphaericus and B. thuringiensis.

In a preferred aspect, the sabadilla alkaloids are derived from Schoenocaulon officinale.

### DETAILED DESCRIPTION OF THE INVENTION

Applicant unexpectedly discovered that pesticidal mixtures of sabadilla alkaloids and Lysinibacillus sphaericus provided enhanced pesticidal activity compared to either pesticide alone. Further, Applicant discovered that pesticidal mixtures of sabadilla alkaloids with a mixture of L. sphaericus and Bacillus thuringiensis provided enhanced pesticidal activity compared to either pesticide alone.

The present invention is directed to pesticidal mixtures of sabadilla alkaloids and a bacterium selected from the group consisting of L. sphaericus and a mixture of L. sphaericus and B. thuringiensis.

Sabadilla alkaloids may be derived from any species of Schoenocaulon. The genus Schoenocaulon includes the following species: S. calcicola, S. caricifolium, S. comatum, S. conzattii, S. dubium (alt. S. gracile), S. framei, S. ghiesbreghtii (alt. S. drummondii, S. yucatanense), S. ignigenum, S. intermedium, S. jaliscense, S. macrocarpum (alt. S. lauricola), S. madidorum, S. megarrhizum, S. mortonii, S. oaxacense, S. obtusum, S. officinale, S. pellucidum, S. plumosum, S. pringlei, S. rzedowskii, S. tenorioi, S. tenue, S. tenuifolium, S. texanum, and S. tigrense. In a preferred embodiment the sabadilla alkaloids are derived from S. officinale. In another preferred embodiment the sabadilla alkaloids are veratridine and cevadine.

Lysinibacillus sphaericus contains six subspecies groups including I, IIA, IIB, III, IV and V. In a preferred embodiment, the L. sphaericus is from the subspecies group IIA.

Bacillus thuringiensis includes many subspecies, each of which are suitable for use in the present invention alone, or in combination. Subspecies of B. thuringiensis include, but are not limited to, aizawai, alesti, berliner, βnitimus, cameroun, canadiensis, colmeri, coreanensis, dakota, darmstadiensis, dendrolimus, entomocidus, fukuokaensis, galleriae, higo, indiana, israelensis, japonensis, japonensis Buibui, jegathesan, kenyae, kumamotoensis, kunthala, kurstaki, kyushuensis, Medellin, mexcanensis, morrisoni, neoleonensis, nigeriae, oloke, ongbei, ostriniae, pakistani, pondicheriensis, roskildiensis, san diego, shandogiensis, shanghai, silo, sotto, subtoxicus, tenebrionis, thompsoni, thuringiensis, tochigiensis, tohokuensis, tolworthi, toumanoffi, wuhanensis, yunnanensis. In a preferred embodiment, B. thuringiensis comprises bacteria of subspecies selected from aizawai, israelensis, kurstaki, thuringiensis and combinations thereof. In a more preferred embodiment, B. thuringiensis comprises bacteria of subspecies selected from aizawai, kurstaki, thuringiensis and combinations thereof. In another preferred embodiment, B. thuringiensis comprises bacteria from a combination of subspecies selected from the group consisting of: aizawai and kurstaki; aizawai and thuringiensis; and kurstaki and thuringiensis.

As used herein, all numerical values relating to amounts, weight percentages and the like are defined as “about” or “approximately” each particular value, namely, plus or minus 10%. For example, the phrase “at least 5% by weight” is to be understood as “at least 4.5% to 5.5% by weight.” Therefore, amounts within 10% of the claimed values are encompassed by the scope of the claims.

As used herein, w/w denotes weight by weight of the total mixture.

The term “effective amount” means the amount of the formulation that will control the target pest. The “effective amount” will vary depending on the mixture concentration, the type of pest(s) being treated, the severity of the pest infestation, the result desired, and the life stage of the pest during treatment, among other factors. Thus, it is not always possible to specify an exact “effective amount.” However, an appropriate “effective amount” in any individual case may be determined by one of ordinary skill in the art.

In a preferred embodiment, the ratio of sabadilla alkaloids to bacterium is from about 1:1,000 to about 1:1, more preferably from about 1:500:1 to about 1:2, yet more preferably from about 1:100 to about 1:3 and most preferably from about 1:76 to about 1:4.

In another preferred embodiment, the pesticidal mixtures of the present invention may contain one or more excipients selected from the group consisting of solvents, anti-caking agents, stabilizers, defoamers, slip agents, humectants, dispersants, wetting agents, thickening agents, emulsifiers, penetrants, adjuvants, polymers, propellants and/or preservatives.

The present invention is further directed to methods of controlling a pest comprising applying a pesticidal mixture comprising an effective amount of sabadilla alkaloids and a bacterium selected from the group consisting of L. sphaericus and a mixture of L. sphaericus and B. thuringiensis to the pest or the pest's environment.

In a preferred embodiment, the pest is selected from a mosquito and a nematode.

In an embodiment, the pest controlled is selected from the group consisting of yellow fever mosquito (Aedes aegypti), southern house mosquito (Culex quinquefasciatus), African malaria mosquito (Anopheles gambiae), common malaria mosquito (Anopheles quadrimaculatus).

The pesticidal mixtures of the present invention can be applied by any convenient means. Those skilled in the art are familiar with the modes of application including spraying, brushing, soaking, in-furrow treatments, pressurized liquids (aerosols), fogging or side-dressing.

In a preferred embodiment, sabadilla alkaloids are applied to the pest or the pest's environment at a rate from about 1 to about 1,000 grams per hectare (“g/HA”), preferably from about 10 to about 700 g/HA and most preferably from about 22 to about 105 g/HA.

In a preferred embodiment, the bacterium is applied to the pest or the pest's environment at a rate from about 10 to 10,000 g/HA, more preferably from about 100 to about 5,000 g/HA, yet more preferably from about 200 to about 2,000 g/HA and most preferably from about 420 to about 1681 g/HA.

In another preferred embodiment, pesticidal mixtures of the present invention comprise from about 0.05% to about 0.5% w/w sabadilla alkaloids.

As used herein, “control” a pest or “controlling” pest(s) refers to killing, incapacitating, repelling, or otherwise decreasing the negative impact of the pest on plants or animals to a level that is desirable to the grower or animal.

As used herein, “pest's environment” refers to any area that the pest is present during any life stage. One environment likely to be treated by the methods of the present invention includes the plants that the pest is living on and the surrounding soil. The pest's environment may also include harvested plants, gardens, fields, greenhouses, or other buildings, and various indoor surfaces and structures, such as furniture including beds, and furnishings including books, clothing, etc.

The articles “a,” “an” and “the” are intended to include the plural as well as the singular, unless the context clearly indicates otherwise. For example, the methods of the present invention are directed to controlling “pest” but this can include control of a multiple pests (such as a more than one insect or more than one insect species).

The following examples are intended to illustrate the present invention and to teach one of ordinary skill in the art how to use the extracts of the invention. They are not intended to be limiting in any way.

### EXAMPLES

### Example 1—Mosquitoes

In this study, the response of the mosquito to application of a 1:76, 1:19, 1:16, and 1:4 ratio of sabadilla (S. officinale) alkaloids to bacterial toxins will be observed. Specifically, sabadilla alkaloids and bacterial toxins will be applied to the pest at the respective rates of: 1) 22 g/HA and 420 g/HA; 2) 105 g/HA and 420 g/HA; 3) 22 g/HA and 1681 g/HA; and 4) 105 g/HA and 1681 g/HA.

The results of the study are predicted to show more than an additive effect. One can determine that the response is more than additive using the following formula: % Cₑₓₚ=A+B−(AB/100).

What is claimed is:

1. A pesticidal mixture comprising an effective amount of sabadilla alkaloids and a bacterium selected from the group consisting of Lysinibacillus sphaericus and a mixture of Lysinibacillus sphaericus and Bacillus thuringiensis.

2. The mixture of claim 1, wherein the sabadilla alkaloids are derived from Schoenocaulon officinale.

3. The mixture of claim 1, wherein the sabadilla alkaloids are veratridine and cevadine.

4. The mixture of claim 1, wherein the bacterium is Lysinibacillus sphaericus.

5. The mixture of claim 1, wherein the bacterium is Lysinibacillus sphaericus subspecies group IIA.

6. The mixture of claim 1, wherein the bacterium is a mixture of Lysinibacillus sphaericus and Bacillus thuringiensis.

7. The mixture of claim 6, wherein the Bacillus thuringiensis is a subspecies selected from the group consisting of kurstaki, israelensis, aizawai and mixtures thereof.

8. A method of controlling a pest comprising applying the pesticidal mixture of claim 1 to the pest or the pest's environment.

9. The method of claim 8, wherein the pest is a mosquito.

10. The method of claim 8, wherein the mosquito is of a genus selected from the group consisting of Aedes, Culex, and Anopheles.

11. The method of claim 8, wherein the pest is selected from the group consisting of yellow fever mosquito (Aedes aegypti), southern house mosquito (Culex quinquefasciatus), African malaria mosquito (Anopheles gambiae), common malaria mosquito (Anopheles quadrimaculatus).

12. The method of claim 8, wherein the sabadilla alkaloid is applied at a rate from about 1 to about 1,000 grams per hectare.

13. The method of claim 8, wherein the sabadilla alkaloid is applied at a rate from about 10 to about 700 grams per hectare.

14. The method of claim 8, wherein the sabadilla alkaloid is applied at a rate from about 22 to about 105 grams per hectare.

15. The method of claim 8, wherein the bacterium is applied at a rate from about 10 to about 10,000 grams per hectare.

16. The method of claim 8, wherein the bacterium is applied at a rate from about 100 to about 5,000 grams per hectare.

17. The method of claim 8, wherein the bacterium is applied at a rate from about 420 to about 1681 grams per hectare.

18. A pesticidal mixture comprising an effective amount of sabadilla alkaloids and a fermentate of a bacterium selected from the group consisting of Lysinibacillus sphaericus and a mixture of Lysinibacillus sphaericus and Bacillus thuringiensis.

19. A method of controlling a pest comprising applying the pesticidal mixture of claim 18 to the pest or the pest's environment.
